(19) United States
(12) Patent Application Publication
(10) Pub. No.: US 2024/0197933 A1
(43) Pub. Date: Jun. 20, 2024

(54) MEDICAL RUBBER PRODUCT

(71) Applicant: Sumitomo Rubber Industries, Ltd., Kobe-shi (JP)

(72) Inventors: Yogun KIDA, Kobe-shi (JP); Kazuki NOJIRI, Kobe-shi (JP); Kei TAJIMA, Kobe-shi (JP); Yusuke SHIBATA, Kobe-shi (JP)

(73) Assignee: Sumitomo Rubber Industries, Ltd., Kobe-shi (JP)

(21) Appl. No.: 18/522,660

(22) Filed: Nov. 29, 2023

(30) Foreign Application Priority Data
Dec. 12, 2022 (JP) 2022-198168

(51) Int. Cl. A61L 2/00; A61M 5/31

(52) U.S. Cl. CPC A61L 2/0035; A61L 2/0058; A61M 5/31; A61M 2005/311

(57) ABSTRACT

An object of the present invention is to provide a sterilization method for a medical rubber product in which non-eluting characteristics are maintained even after sterilization with gamma ray and with which less troubles occur in a medical product manufacturing process. The medical rubber product according to the present invention is a medical rubber product formed from an elastic material and sterilized by being irradiated with gamma ray or electron ray (beta ray), wherein, when measurement is performed on a surface portion of the elastic material through ATR by using an FT-IR, if an area of an infrared absorption peak at around a wave number of 1650 cm⁻¹ in an infrared absorption spectrum obtained through the measurement is defined as As and an area of an infrared absorption peak at around a wave number of 1470 cm⁻¹ in the infrared absorption spectrum is defined as Bs, Ss=(As/Bs)×100≤6 is satisfied.

### CROSS-REFERENCE TO RELATED APPLICATION

The present application claims priority to Japanese Patent Application No. 2022-198168, filed on Dec. 12, 2022, the entire contents of which are incorporated herein by reference.

### BACKGROUND

### Field

The present disclosure relates to a medical rubber product and more specifically relates to a sterilized medical rubber product.

### Background Art

Medical rubber plugs for tightly closing openings of syringes, vials, and the like may be required to have many characteristics such as non-eluting characteristics, high cleanability, chemical resistance, resistance to needle piercing, self-sealability, and high slidability. Quality characteristics that may be required of the medical rubber plugs should, in terms of use of the medical rubber plugs, comply with the regulations stipulated in “Test for Rubber Closure for Aqueous Infusions” of the 17th edition of the Japanese Pharmacopoeia, as an example.

For example, Japanese Laid-Open Patent Publication No. H10-179690 describes a rubber plug for a pharmaceutical agent container, the rubber plug being characterized by being obtained by: blending 5 to 25 parts by weight of fine powder of polyethylene with an ultrahigh molecular weight per 100 parts by weight of a halogenated isobutylene-isoprene rubber; and vulcanizing the resultant halogenated isobutylene-isoprene rubber by using at least one of 2-substituted-4,6-dithiol-s-triazine derivatives or by using an organic peroxide, in the absence of a zinc compound.

There is an increasing demand for medical rubber products (syringe gaskets, vial plugs, and the like) to be delivered in a state of guaranteeing sterilization thereof, i.e., to be ready-to-use (RTU). As methods for guaranteeing sterilization, there are methods involving sterilization with high-pressure steam, sterilization with ethylene oxide gas (EOG), and sterilization with gamma ray. The method involving sterilization with gamma ray can involve a medical rubber product being sterilized in a state of being packaged and thus can be delivered without opening the package. Meanwhile, the method involving sterilization with EOG may have environment-related issues. In view of this, the method for guaranteeing sterilization may tend to be switched to the method involving sterilization with gamma ray.

The method involving sterilization with gamma ray may guarantee sterilization by means of absorbed dose setting and actually measured values. If a plurality of medical rubber products are put into a packaging bag and sterilization with gamma ray is performed, unevenness among the medical rubber products might occur in the packaging bag. Thus, even when the packaging bag is irradiated with a predetermined radiation dose of gamma ray, variation in the absorbed dose of gamma ray may occur in the packaging bag. This may give rise to: medical rubber products having low absorbed doses of gamma ray; and medical rubber products having high absorbed doses of gamma ray. However, it may be necessary to ensure, for each medical rubber product, a minimum absorbed dose with which the medical rubber product can be sterilized. Thus, it may be necessary to irradiate the packaging bag with at least the minimum absorbed dose of gamma ray. This can give rise to medical rubber products that absorb an excessive dose of gamma ray at the time of sterilization with gamma ray, in the packaging bag.

Japanese Laid-Open Patent Publication No. 2002-301133 describes: a rubber composition containing an isobutylene copolymer as a main component and having a density not higher than 0.95, the rubber composition being for use in a medical rubber plug or a medical rubber product on which radiation treatment is easily performed; and a crosslinked product of the rubber composition.

Japanese Laid-Open Patent Publication (Translation of PCT Application) No. 2017-531604 describes a method for packaging a part (1) made from an elastomer, such as a plug for a pharmaceutical agent container. The method includes: a step of packing the part (1) into a primary bag (10) made from a material substantially impermeable with air; and a step of applying an atmosphere with at least 80% of nitrogen to the inside of the primary bag (10). In the method, the primary bag (10) is put into a secondary bag (20), and the interval between the primary bag (10) and the secondary bag (20) is set to be in a vacuum state.

When a medical rubber product is sterilized by being irradiated with gamma ray, cleavage and crosslinking can simultaneously occur in a polymer that forms the medical rubber product. If an excessive dose of gamma ray is absorbed, cleavage of the main chain of the polymer that forms the medical rubber product may be promoted, whereby low-molecular-weight components may be generated. Consequently, the eluting performance of the medical rubber product having been sterilized with gamma ray may deteriorate. In addition, bleed-out, onto a surface of the rubber product, of the low-molecular-weight components resulting from the cleavage may occur, and medical rubber products may come into close contact with each other. Consequently, a trouble of clogging in a parts feeder used in a medical product manufacturing process may occur.

It is also conceivable to blend an antioxidant to the medical rubber product in preparation for absorption of an excessive dose of gamma ray. However, addition of an antioxidant may lead to concerns that: eluting characteristics may decrease; and a medical preparation may be adversely influenced.

### SUMMARY

A medical rubber product according to an aspect of the present disclosure can be a medical rubber product formed from an elastic material and sterilized by being irradiated with gamma ray or electron ray, wherein, when measurement is performed on a surface portion of the elastic material through attenuated total reflection (ATR) by using a Fourier transform infrared spectrophotometer (FT-IR), if an area of an infrared absorption peak at around a wave number of 1650 cm⁻¹ in an infrared absorption spectrum obtained through the measurement is defined as As and an area of an infrared absorption peak at around a wave number of 1470 cm⁻¹ in the infrared absorption spectrum is defined as Bs, Ss=(As/Bs)×100≤6 is satisfied.

### BRIEF DESCRIPTION OF THE DRAWINGS

FIG. 1 is a diagram for schematically explaining an example of a packaging mode for medical rubber products according to one or more embodiments of the present disclosure;

FIG. 2 is a diagram for schematically explaining another example of the packaging mode for the medical rubber products according to one or more embodiments of the present disclosure;

FIG. 3 is a chart showing results of FT-IR measurement performed on a medical rubber product according to one or more embodiments of the present disclosure;

FIG. 4 is a chart showing results of FT-IR measurement performed on a medical rubber product in a comparative example; and

FIG. 5 is a diagram for schematically explaining a tack test method for the medical rubber product according to one or more embodiments of the present disclosure.

### DETAILED DESCRIPTION

One or more embodiments of present disclosure have been made in view of the above circumstances in the Background section, and an object of one or more embodiments of the present disclosure, among one or more objects, can be to provide a sterilization method for a medical rubber product in which non-eluting characteristics can be maintained even after sterilization with gamma ray.

One or more embodiments of present disclosure can make it possible to provide a medical rubber product in which non-eluting characteristics can be maintained even after sterilization with gamma ray and with which less troubles may occur in a medical product manufacturing process.

A medical rubber product according to one or more embodiments of the present disclosure can be a medical rubber product formed from an elastic material and sterilized by being irradiated with gamma ray or electron ray, wherein, when measurement is performed on a surface portion of the elastic material through attenuated total reflection (ATR) by using a Fourier transform infrared spectrophotometer (FT-IR), in the case that an area of an infrared absorption peak at around a wave number of 1650 cm⁻¹ in an infrared absorption spectrum obtained through the measurement is defined as As and an area of an infrared absorption peak at around a wave number of 1470 cm⁻¹ in the infrared absorption spectrum is defined as Bs, Ss=(As/Bs)×100≤6 can be satisfied.

The medical rubber product according to one or more embodiments of the present can be a medical rubber product sterilized by being irradiated with gamma ray or electron ray. An infrared absorption spectrum (vertical axis: absorbance, horizontal axis: wave number) can be obtained through measurement on the surface portion of the elastic material that forms the medical rubber product, where the measurement can be performed through attenuated total reflection (ATR) by using a Fourier transform infrared spectrophotometer (FT-IR).

As the surface portion of the medical rubber product, a measurement sample can be made by being cut out from the surface of the rubber product, for instance, with use of a razor, so as to have a thickness (for example, 0.5 mm to 2.0 mm) that can allow the measurement sample to be placed on a measurement device. The measurement sample can be placed on the measurement device, and a region, of a surface portion of the measurement sample, having an outer diameter of 1 mm, for instance, can be irradiated with light and can be subjected to measurement.

In the infrared absorption spectrum, an infrared absorption peak A having an absorption peak of absorbance at around a wave number of 1650 cm⁻¹ can be present. The infrared absorption peak can correspond to a carbonyl (C═O stretching vibration) group (a ketone, an aldehyde, a carboxylic acid, an ester, or the like) attributed to an oxide of a polymer composing the elastic material. The phrase “at around a wave number of 1650 cm⁻¹” can mean a region ranging over wave numbers of 1650 cm⁻¹±100 cm⁻¹ and more preferably can mean a region ranging over wave numbers of 1650 cm⁻¹±50 cm⁻¹.

In the infrared absorption spectrum, an infrared absorption peak B having an absorption peak of absorbance at around a wave number of 1470 cm⁻¹ can be present. The infrared absorption peak B can be attributed to scissoring vibration (in-plane deformation vibration) of —CH₂— in the main chain of the polymer composing the elastic material. The phrase “at around a wave number of 1470 cm⁻¹” can mean a region ranging over wave numbers of 1470 cm⁻¹±50 cm⁻¹ and more preferably can mean a region ranging over wave numbers of 1470 cm⁻¹±20 cm⁻¹.

The surface portion of the elastic material that forms the medical rubber product according to one or more embodiments of the present disclosure can be as follows. That is, in the case that the area of the infrared absorption peak A is defined as As and the area of the infrared absorption peak B is defined as Bs, a value Ss calculated according to Ss=(As/Bs)×100 can be not larger than 6, preferably not larger than 5.5, or preferably not larger than 5.0. If the value Ss is not larger than 6, degradation of the polymer composing the elastic material of the medical rubber product can be suppressed, and the medical rubber product can come to have excellent non-eluting characteristics. Meanwhile, since the surface portion of the elastic material is oxidized during manufacturing and processing of the medical rubber product, the value Ss can be generally larger than 3.0 and more generally not smaller than 3.5.

Cut-out from a center portion of the medical rubber product can be performed and measurement can be performed on an exposed inside of the medical rubber product through attenuated total reflection (ATR) by using the Fourier transform infrared spectrophotometer (FT-IR), whereby an infrared absorption spectrum (vertical axis: absorbance, horizontal axis: wave number) can be obtained.

Specifically, the medical rubber product can be cut in the longitudinal direction through the center thereof in a plan view. A measurement sample can be obtained by being cut out from a center portion of the obtained cross section with use of the razor so as to have a thickness (for example, 0.5 mm to 2.0 mm) that can allow the measurement sample to be placed on the measurement device. The measurement sample can be placed on the measurement device, and a region, of a surface portion of the measurement sample (the center portion of the cross section), having an outer diameter of 1 mm can be irradiated with light and can be subjected to measurement.

In the infrared absorption spectrum, an infrared absorption peak A having an absorption peak of absorbance at around a wave number of 1650 cm⁻¹ can be present. The infrared absorption peak can be attributed to the oxide of the polymer composing the elastic material. The phrase “at around a wave number of 1650 cm⁻¹” can mean a region ranging over wave numbers of 1650 cm⁻¹±100 cm⁻¹ and more preferably can mean a region ranging over wave numbers of 1650 cm⁻¹±50 cm⁻¹.

In the infrared absorption spectrum, an infrared absorption peak B having an absorption peak of absorbance at around a wave number of 1470 cm⁻¹ can be present. The infrared absorption peak B can be attributed to scissoring vibration (in-plane deformation vibration) of —CH₂— in the main chain of the polymer composing the elastic material. The phrase “at around a wave number of 1470 cm⁻¹” can mean a region ranging over wave numbers of 1470 cm⁻¹±50 cm⁻¹ and more preferably can mean a region ranging over wave numbers of 1470 cm⁻¹±20 cm⁻¹.

The inside of the elastic material that forms the medical rubber product according to one or more embodiments of the present disclosure can be as follows. That is, in the case that the area of the infrared absorption peak A is defined as Ai, the area of the infrared absorption peak B is defined as Bi, and Si=(Ai/Bi)×100 is satisfied, a value calculated according to P=(Ss/Si)×100 can be not larger than 150, preferably not larger than 140, or preferably not larger than 130. The value P (=(Ss/Si)×100) can be an index of the extent of degradation at the inside and the surface portion of the medical rubber product. If the value P is not larger than 150, the extent of degradation at the surface portion may be small, and non-eluting characteristics and non-adhesiveness can be favorable. The lower limit of the value P may not be particularly limited, according to one or more embodiments of the present disclosure.

### [Medical Rubber Product]

The medical rubber product according to one or more embodiments of the present disclosure can be formed from an elastic material. The elastic material may not be particularly limited, for instance, as long as the elastic material contains a polymer having a methylene chain (—CH₂—) in the main chain thereof. The elastic material can be composed of a rubber component, according to one or more embodiments of the present disclosure.

The elastic material can be a cured product of a medical rubber composition containing, comprising, or consisting of: a (a) base polymer containing a halogenated isobutylene-isoprene rubber; and a triazine derivative as a (c) crosslinking agent. The elastic material can be a cured product of a medical rubber composition containing, comprising, or consisting of: a (a) base polymer containing a halogenated isobutylene-isoprene rubber; a (b) polyethylene; and a triazine derivative as a (c) crosslinking agent. Hereinafter, raw materials in the medical rubber composition according to one or more embodiments of the present disclosure will be described.

First, the (a) base polymer containing, comprising, or consisting of a halogenated isobutylene-isoprene rubber will be described. Examples of the halogenated isobutylene-isoprene rubber can include: chlorinated isobutylene-isoprene rubber; brominated isobutylene-isoprene rubber; a bromide of a copolymer rubber of isobutylene and p-methylstyrene (brominated isobutylene-para-methylstyrene copolymer rubber); and the like.

As the halogenated isobutylene-isoprene rubber, chlorinated isobutylene-isoprene rubber or brominated isobutylene-isoprene rubber may be preferable. The chlorinated isobutylene-isoprene rubber or the brominated isobutylene-isoprene rubber can be obtained by, for example, causing a reaction in which: chlorine or bromine is added to an isoprene structural moiety (specifically, a double bond and/or a carbon atom adjacent to the double bond) in an isobutylene-isoprene rubber; or the isoprene structural moiety is substituted with chlorine or bromine. The isobutylene-isoprene rubber can be a copolymer obtained by polymerizing isobutylene and a small amount of isoprene.

The halogen content of the halogenated isobutylene-isoprene rubber can be preferably not lower than 0.5% by mass, more preferably not lower than 1% by mass, and further preferably not lower than 1.5% by mass. Meanwhile, the halogen content can be preferably not higher than 5% by mass, more preferably not higher than 4% by mass, and further preferably not higher than 3% by mass.

Specific examples of the chlorinated isobutylene-isoprene rubber can include at least one of: CHLOROBUTYL 1066 [stabilizer: NS, halogen content: 1.26%, Mooney viscosity: 38 ML₁₊₈ (125° C.), specific gravity: 0.92] manufactured by JAPAN BUTYL Co., Ltd.; LANXESS X_BUTYL CB1240 manufactured by LANXESS; and/or the like.

Specific examples of the brominated isobutylene-isoprene rubber can include at least one of: BROMOBUTYL 2255 [stabilizer: NS, halogen content: 2.0%, Mooney viscosity: 46 ML₁₊₈ (125° C.), specific gravity: 0.93] manufactured by JAPAN BUTYL Co., Ltd.; LANXESS X_BUTYL BBX2 manufactured by LANXESS; and/or the like.

The (a) base polymer may contain, comprise, or consist of a rubber component other than the halogenated isobutylene-isoprene rubber.

Examples of the other rubber component can include butyl-based rubbers, isoprene rubber, butadiene rubber, styrene-butadiene rubber, natural rubber, chloroprene rubber, nitrile-based rubbers such as acrylonitrile-butadiene rubber, hydrogenated nitrile-based rubbers, norbornene rubber, ethylene-propylene rubber, ethylene-propylene-diene rubber, acrylic rubber, ethylene-acrylate rubber, fluororubber, chlorosulfonated polyethylene rubber, epichlorohydrin rubber, silicone rubber, urethane rubber, polysulfide rubber, phosphazene rubber, 1,2-polybutadiene, and the like. These rubber components may be used singly, or two or more of these rubber components may be used in combination.

In the case of using the other rubber component, the proportion of the halogenated isobutylene-isoprene rubber in the (a) base polymer can be preferably not lower than 90% by mass, more preferably not lower than 95% by mass, and further preferably not lower than 98% by mass. A mode in which the (a) base polymer can contains or comprise only the halogenated isobutylene-isoprene rubber may also be preferable, according to one or more embodiments of the present disclosure.

The medical rubber composition according to one or more embodiments of the present can preferably contain, comprise, or consist of the (b) polyethylene. The polyethylene can be more likely to absorb gamma ray than the (a) base polymer. Thus, the polyethylene can have an effect of preventing cleavage of a chain of the (a) base polymer due to irradiation with gamma ray. In addition, a polyethylene having a low degree of crystallinity can have a branched chain, and it can be considered that crosslinking progresses without cleavage of the main chain even upon irradiation with gamma ray. As a result, the eluting performance of the medical rubber composition can be considered to be improved.

From this viewpoint, examples of the (b) polyethylene to be used in one or more embodiments of the present disclosure can include high-density polyethylene (HDPE) and low-density polyethylene (LDPE). The high-density polyethylene (HDPE) and the low-density polyethylene (LDPE) may be used singly, or the high-density polyethylene (HDPE) and the low-density polyethylene (LDPE) may be used in combination.

In the case of using the high-density polyethylene (HDPE) and the low-density polyethylene (LDPE) in combination, the mass ratio (HDPE/LDPE) of the high-density polyethylene (HDPE) to the low-density polyethylene (LDPE) can be preferably not lower than 0.3, more preferably not lower than 0.5, and further preferably not lower than 1.0. Meanwhile, the mass ratio (HDPE/LDPE) can be preferably not higher than 5.0, more preferably not higher than 4.0, and further preferably not higher than 3.0. This can be because, if the mass ratio (HDPE/LDPE) of the high-density polyethylene (HDPE) to the low-density polyethylene (LDPE) falls within the aforementioned range, a radical absorption effect at the time of irradiation with gamma ray and an appropriate hardness of the rubber can be ensured.

The (b) polyethylene can contain, comprise, or consist of a polyethylene having a degree of crystallinity not higher than 70%, according to one or more embodiments of the present disclosure.

The degree of crystallinity of the high-density polyethylene (HDPE) can be preferably 60% to 80%, more preferably 60% to 75%, and further preferably 60% to 70%. The degree of crystallinity of the low-density polyethylene (LDPE) can be preferably 30% to 50%, more preferably 30% to 45%, and further preferably 30% to 40%. This can be because, if the degree of crystallinity of each polyethylene falls within the aforementioned corresponding range, radicals generated through irradiation with gamma ray can be effectively absorbed, and cleavage of the main chain of the polymer can be prevented.

The degree of crystallinity of the (b) polyethylene can be determined according to the following expression.

Degree of crystallinity (%)=(measured melting heat quantity (J/g)/perfect crystal melting heat quantity (J/g))×100

The perfect crystal melting heat quantity (J/g) is 293 J/g (a value in a literature) and can be a melting heat quantity of the polyethylene at 100% crystallinity. A measurement method for the melting heat quantity of the polyethylene will be described later.

As the (b) polyethylene, the low-density polyethylene may be preferable. The density (g/cm³) of the high-density polyethylene can be preferably 0.930 to 0.960 and more preferably 0.930 to 0.950. The density (g/cm³) of the low-density polyethylene may not be particularly limited, but can be preferably 0.910 to 0.925 and more preferably 0.910 to 0.920.

As the (b) polyethylene, a polyethylene in the form of fine powder can be preferably used. The volume-average particle diameter of the polyethylene in the form of fine powder can be preferably not smaller than 10 μm, more preferably not smaller than 15 μm, and further preferably not smaller than 20 μm. Meanwhile, the volume-average particle diameter can be preferably not larger than 200 μm, more preferably not larger than 160 μm, and further preferably not larger than 120 μm. This can be because, if the particle diameter of the polyethylene in the form of fine powder falls within the aforementioned range, it can become easy for the polyethylene to be evenly mixed or dispersed in the polymer.

The blending amount of the (b) polyethylene per 100 parts by mass of the (a) base polymer can be preferably not smaller than 3 parts by mass, more preferably not smaller than 5 parts by mass, and further preferably not smaller than 10 parts by mass. Meanwhile, the blending amount can be preferably not larger than 30 parts by mass, more preferably not larger than 25 parts by mass, and further preferably not larger than 20 parts by mass. This can be because, if the blending amount of the (b) polyethylene falls within the aforementioned range, radicals generated at the time of irradiation with gamma ray can be effectively absorbed, and cleavage of the main chain of the polymer can be prevented.

The medical rubber composition according to one or more embodiments of the present disclosure can preferably contain, be comprised of, or consist of a triazine derivative as the (c) crosslinking agent.

The triazine derivative can act as a crosslinking agent on the halogenated isobutylene-isoprene rubber. Examples of the triazine derivative can include a compound represented by a general formula (1).

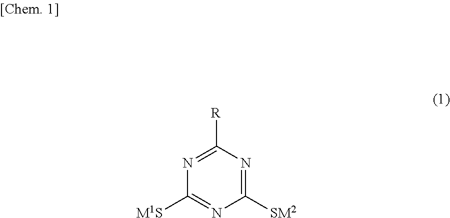

[in the formula, R represents —SH, —OR¹, —SR², —NHR³, or —NR⁴R⁵ (R¹, R², R³, R⁴, and R⁵ each represent an alkyl group, an alkenyl group, an aryl group, an aralkyl group, an alkylaryl group, or a cycloalkyl group. R⁴ and R⁵ may be identical to each other or different from each other.). M¹ and M² each can represent H, Na, Li, K, ½Mg, ½Ba, ½Ca, an aliphatic primary, secondary, or tertiary amine, a quaternary ammonium salt, or a phosphonium salt. M¹ and M² may be identical to each other or different from each other.]

In the general formula (1), examples of the alkyl group can include alkyl groups having 1 to 12 carbon atoms, such as a methyl group, an ethyl group, an n-propyl group, an isopropyl group, an n-butyl group, an isobutyl group, a tert-butyl group, an n-pentyl group, an isopentyl group, a tert-pentyl group, an n-hexyl group, a 1,1-dimethylpropyl group, an octyl group, an isooctyl group, a 2-ethylhexyl group, a decyl group, and a dodecyl group. Examples of the alkenyl group can include alkenyl groups having 1 to 12 carbon atoms, such as a vinyl group, an allyl group, a 1-propenyl group, an isopropenyl group, a 2-butenyl group, a 1,3-butadienyl group, and a 2-pentenyl group. Examples of the aryl group can include monocyclic aromatic hydrocarbon groups and condensed polycyclic aromatic hydrocarbon groups, and examples thereof include: aryl groups having 6 to 14 carbon atoms, such as a phenyl group, a naphthyl group, an anthryl group, a phenanthryl group, and an acenaphthylenyl group; and the like. Examples of the aralkyl group can include aralkyl groups having 7 to 19 carbon atoms, such as a benzyl group, a phenethyl group, a diphenylmethyl group, a 1-naphthylmethyl group, a 2-naphthylmethyl group, a 2,2-diphenylethyl group, a 3-phenylpropyl group, a 4-phenylbutyl group, a 5-phenylpentyl group, a 2-biphenylylmethyl group, a 3-biphenylylmethyl group, and a 4-biphenylylmethyl group. Examples of the alkylaryl group include alkylaryl groups having 7 to 19 carbon atoms, such as a tolyl group, a xylyl group, and an octylphenyl group. Examples of the cycloalkyl group can include: cycloalkyl groups having 3 to 9 carbon atoms, such as a cyclopropyl group, a cyclobutyl group, a cyclopentyl group, a cyclohexyl group, a cycloheptyl group, a cyclooctyl group, and a cyclononyl group; and the like.

Specific examples of the triazine derivative can be represented by the general formula (1) and can include 2,4,6-trimercapto-s-triazine, 2-methylamino-4,6-dimercapto-s-triazine, 2-(n-butylamino)-4,6-dimercapto-s-triazine, 2-octylamino-4,6-dimercapto-s-triazine, 2-propylamino-4,6-dimercapto-s-triazine, 2-diallylamino-4,6-dimercapto-s-triazine, 2-dimethylamino-4,6-dimercapto-s-triazine, 2-dibutylamino-4,6-dimercapto-s-triazine, 2-di(iso-butylamino)-4,6-dimercapto-s-triazine, 2-dipropylamino-4,6-dimercapto-s-triazine, 2-di(2-ethylhexyl)amino-4,6-dimercapto-s-triazine, 2-dioleylamino-4,6-dimercapto-s-triazine, 2-laurylamino-4,6-dimercapto-s-triazine, 2-anilino-4,6-dimercapto-s-triazine, and sodium salts and disodium salts of these triazine derivatives.

Among these triazine derivatives, 2,4,6-trimercapto-s-triazine, 2-dialkylamino-4,6-dimercapto-s-triazine, and 2-anilino-4,6-dimercapto-s-triazine are preferable, and 2-dibutylamino-4,6-dimercapto-s-triazine can be particularly preferable since 2-dibutylamino-4,6-dimercapto-s-triazine may be relatively easy to obtain.

Other examples of the triazine derivative can include one or more of 6-[bis(2-ethylhexyl)amino]-1,3,5-triazine-2,4-dithiol, 6-diisobutylamino-1,3,5-triazine-2,4-dithiol, 6-dibutylamino-1,3,5-triazine-2,4-dithiol, 6-dibutylamino-1,3,5-triazine-2,4-dithiol monosodium, 6-anilino-1,3,5-triazine-2,4-dithiol, 1,3,5-triazine-2,4,6-trithiol, and/or the like.

According to one or more embodiments of the present disclosure, these triazine derivatives may be used singly, or two or more of these triazine derivatives may be used in combination.

In the medical rubber composition according to one or more embodiments of the present disclosure, the amount of the (c) triazine derivative contained per 100 parts by mass of the (a) base polymer component can be preferably not smaller than 0.1 parts by mass, more preferably not smaller than 0.3 parts by mass, and further preferably not smaller than 0.5 parts by mass. Meanwhile, the amount can be preferably not larger than 2.0 parts by mass, more preferably not larger than 1.4 parts by mass, and further preferably not larger than 1.2 parts by mass. This an be because, if the amount of the (c) triazine derivative contained falls within the aforementioned range, a rubber having favorable rubber physical properties (hardness, tensile properties, Cset) and good eluting performance and processability (less susceptibility to scorching) can be obtained.

The medical rubber composition according to one or more embodiments of the present disclosure can preferably contain no vulcanization accelerator. This can be because a vulcanization accelerator could remain in a rubber product obtained as a final product and could elute into a drug solution inside a syringe or a vial. Examples of the vulcanization accelerator can include guanidine-based accelerators (e.g., diphenylguanidine), thiuram-based accelerators (e.g., tetramethylthiuram disulfide and tetramethylthiuram monosulfide), dithiocarbamate-based accelerators (e.g., zinc dimethyldithiocarbamate), thiazole-based accelerators (e.g., 2-mercaptobenzothiazole and dibenzothiazyl disulfide), and sulfenamide-based accelerators (N-cyclohexyl-2-benzothiazole sulfenamide and N-t-butyl-2-benzothiazole sulfenamide).

The medical rubber composition according to one or more embodiments of the present disclosure may contain a hydrotalcite. The hydrotalcite can function as an anti-scorching agent upon crosslinking in the halogenated isobutylene-isoprene rubber and can also have a function of preventing increase in compression set in the medical rubber product. Further, the hydrotalcite can function also as an acid acceptor for absorbing chlorine-based gas and bromine-based gas, which have been generated upon crosslinking in the halogenated isobutylene-isoprene rubber, and preventing occurrence of, for example, crosslinking inhibition due to these gases. Magnesium oxide can also function as an acid acceptor.

Examples of the hydrotalcite can include one or more of Mg—Al-based hydrotalcites such as Mg_{4.5}Al₂(OH)₁₃CO₃·3.5H₂O, Mg_{4.5}Al₂(OH)₁₃CO₃, Mg₄Al₂(OH)₁₂CO₃·3.5H₂O, Mg₆Al₂(OH)₁₆CO₃·4H₂O, Mg₅Al₂(OH)₁₄CO₃·4H₂O, and Mg₃Al₂(OH)₁₀CO₃·1.7H₂O, and the like.

Specific examples of the hydrotalcite include DHT-4A (registered trademark)-2 manufactured by Kyowa Chemical Industry Co., Ltd., and/or the like.

In the case where the hydrotalcite is used as an acid acceptor in the medical rubber composition according to one or more embodiments of the present disclosure, the hydrotalcite can be preferably used in combination with MgO. In this case, the blending amount of the hydrotalcite can be preferably considered in terms of the total amount of the acid acceptors (hydrotalcite and MgO). The total amount of the acid acceptors (hydrotalcite and MgO) contained per 100 parts by mass of the (a) base polymer component can be preferably not smaller than 0.5 parts by mass and more preferably not smaller than 1 part by mass. Meanwhile, the total amount can be preferably not larger than 15 parts by mass and more preferably not larger than 10 parts by mass. This can be because, if the total amount of the acid acceptors (hydrotalcite and MgO) falls within the aforementioned range, generation of rust on a mold or the like can be suppressed, and defects that raw materials themselves turn into an unwanted object in the form of a white spot can be reduced.

The medical rubber composition according to one or more embodiments of the present disclosure may contain a co-crosslinking agent. The co-crosslinking agent can be preferably a polyfunctional (meth)acrylate compound. The polyfunctional (meth)acrylate compound can be more preferably a difunctional or higher-functional (meth)acrylate-based compound and further preferably can be a trifunctional or higher-functional (meth)acrylate-based compound. Meanwhile, the polyfunctional (meth)acrylate compound can be preferably an octafunctional or lower-functional (meth)acrylate-based compound and more preferably a hexafunctional or lower-functional (meth)acrylate-based compound. Examples of the difunctional or higher-functional (meth)acrylate compound can include a compound having at least two acryloyl groups and/or methacryloyl groups. The term “(meth)acrylate” can mean “acrylate” and/or “methacrylate.”

Examples of the difunctional or higher-functional (meth)acrylate-based compound can include di(meth)acrylate of polyethylene glycol, 1,4-butanediol di(meth)acrylate, 1,6-hexanediol di(meth)acrylate, 1,9-nonanediol di(meth)acrylate, trimethylolpropane tri(meth)acrylate, trimethylolethane tri(meth)acrylate, pentaerythritol tri(meth)acrylate, pentaerythritol tetra(meth)acrylate, glycerin tri(meth)acrylate, dipentaerythritol tri(meth)acrylate, dipentaerythritol tetra(meth)acrylate, dipentaerythritol penta(meth)acrylate, dipentaerythritol hexa(meth)acrylate, tripentaerythritol tetra(meth)acrylate, tripentaerythritol penta(meth)acrylate, tripentaerythritol hexa(meth)acrylate, tripentaerythritol hepta(meth)acrylate, and the like. These co-crosslinking agents may be used singly, or two or more of these co-crosslinking agents may be used in combination.

In the medical rubber composition according to one or more embodiments of the present disclosure, a (d) filler may be blended. Examples of the (d) filler can include inorganic fillers such as silica, clay, and talc, as examples. The filler can be further preferably clay or talc. The filler can have a function of adjusting the rubber hardness of the medical rubber product and can function also as an extender for reducing manufacturing cost for the medical rubber product.

Examples of the clay can include calcined clay and kaolin clay. Specific examples of the clay can include SILLITIN (registered trademark) Z manufactured by Hoffmann Mineral GmbH, SATINTONE (registered trademark) W manufactured by Engelhard Corporation, NN Kaolin Clay manufactured by Tsuchiya Kaolin Industry Co., Ltd., PoleStar200R manufactured by Imerys Specialties Japan Co., Ltd., and the like.

Specific examples of the talc can include High toron A manufactured by Takehara Kagaku Kogyo Co., Ltd., MICRO ACE (registered trademark) K-1 manufactured by Nippon Talc Co., Ltd., MISTRON (registered trademark) Vapor manufactured by Imerys Specialties Japan Co., Ltd., and the like.

In the medical rubber composition according to one or more embodiments of the present disclosure, a colorant such as titanium oxide or carbon black, polyethylene glycol as a processing aid or as a crosslinking activator, a plasticizer (for example, paraffin oil), and the like may further be blended in appropriate proportions.

The medical rubber composition according to one or more embodiments of the present disclosure can be obtained by kneading the (a) base polymer containing, comprising, or consisting of the halogenated isobutylene-isoprene rubber, the (b) polyethylene, the triazine derivative as the (c) crosslinking agent, and other blending materials to be added as necessary. The kneading can be performed by using, for example, an open roll, a sealed-type kneader, or the like. The kneaded product can be preferably molded in the shape of a ribbon, the shape of a sheet, the shape of a pellet, or the like, and can be more preferably molded in the shape of a sheet.

If the kneaded product having the shape of a ribbon, the shape of a sheet, or the shape of a pellet is press-molded, a medical rubber product having a desired shape can be obtained. A crosslinking reaction in the medical rubber composition can progress during the pressing. The temperature in the molding can be, for example, preferably not lower than 130° C. and more preferably not lower than 140° C. Meanwhile, the temperature can be preferably not higher than 200° C. and more preferably not higher than 190° C. The time for the molding can be preferably not shorter than 2 minutes and more preferably not shorter than 3 minutes. Meanwhile, the time is preferably not longer than 60 minutes and more preferably not longer than 30 minutes. The pressure for the molding can be preferably not lower than 0.1 MPa and more preferably not lower than 0.2 MPa. Meanwhile, the pressure can be preferably not higher than 10 MPa and more preferably not higher than 8 MPa.

Unnecessary portions can be cut off and removed from the molded product after the press-molding, such that the molded product has a predetermined shape. The obtained molded product can be washed, dried, and packaged to manufacture the medical rubber product.

The JIS-A hardness of the elastic material that forms the medical rubber product according to one or more embodiments of the present disclosure can be preferably not lower than 30, more preferably not lower than 35, and further preferably not lower than 40. Meanwhile, the JIS-A hardness can be preferably not higher than 70, more preferably not higher than 65, and further preferably not higher than 60. This can be because, if the JIS-A hardness of the elastic material falls within the aforementioned range, both product performances such as sealing performance and/or resistance to needle piercing which may be required of rubber plugs can be achieved.

The elastic material that forms the medical rubber product according to one or more embodiments of the present disclosure can have a compression set measured under conditions of 70° C., 22 hours, and 25% according to JIS K 6262. The compression set can be preferably not higher than 20%, more preferably not higher than 15%, and further preferably not higher than 10%. This can be because, if the compression set of the elastic material falls within the aforementioned range, sealing performance required of rubber plugs can be achieved, and a needle hole can be swiftly closed at the time of needle removal after needle piercing, whereby no liquid leakage may occur.

### [Sterilization Method]

The medical rubber product according to one or more embodiments of the present disclosure can be preferably sterilized by being irradiated with a radioactive ray and can be more preferably sterilized by being irradiated with gamma ray or electron ray. A method for the sterilization can preferably include irradiating a packaging article for the medical rubber product with a radioactive ray, where the packaging article can accommodate a plurality of the medical rubber products.

Examples of the radioactive ray to be used for the sterilization can include α-ray (the atomic nucleus of helium), β-ray (electron ray), and γ-ray (gamma ray). β-ray (electron ray) may have a very high dose rate (e.g., several tens of thousands of times the dose rate of γ-ray), and thus the sterilization time may be short. However, since β-ray can be regarded as a particle beam, the penetrating power thereof may be relatively small. Meanwhile, gamma ray can have a large penetrating power but may have a lower dose rate than electron ray, and thus the processing time may be elongated. According to one or more embodiments of the present disclosure, the packaging article, for the medical rubber product, accommodating a plurality of the medical rubber products can be preferably sterilized with gamma ray from the viewpoint of sterilizing the packaging article.

Examples of the gamma ray can include gamma rays emitted from cobalt-60, cesium-137, and the like.

Regarding irradiation with the gamma ray, an absorbed dose of the gamma ray by the medical rubber product can be set through an actual sterilization validation procedure. For ordinary medical devices, operation can be performed with a minimum absorbed dose being set, for instance, to 15 kGy in many cases. A gamma ray radiation dose at which the absorbed doses of the gamma ray by all of the medical rubber products in the packaging article take values not lower than 15 kGy, can vary depending on the number of the medical rubber products in the packaging article, how the medical rubber products are present in the packaging article, and the like. In general, irradiation can be performed in a dose that falls within a range of not lower than 1.4 times 15 kGy and not higher than 2.0 times 15 kGy. Likewise, in the case that the minimum absorbed dose is set to 20 kGy, irradiation can be performed in a dose that falls within a range of not lower than 1.4 times 20 kGy and not higher than 2.0 times 20 kGy, and, in the case that the minimum absorbed dose is set to 25 kGy, irradiation can be performed in a dose that falls within a range of not lower than 1.4 times 25 kGy and not higher than 2.0 times 25 kGy. The absorbed dose of the gamma ray can be ascertained through attachment of a dosemeter to an object that is to be irradiated.

The packaging article accommodating the medical rubber products not having yet been irradiated with the gamma ray can have an oxygen concentration that can be preferably not higher than 5%, more preferably not higher than 3%, and further preferably not higher than 1%. This can be because, if the oxygen concentration in the packaging article is set to be not higher than 5%, degradation of each medical rubber product due to irradiation with the gamma ray can be suppressed.

Examples of a method for setting the oxygen concentration in the packaging article to be not higher than 5% can include: a method in which air in the packaging article is substituted with an inert gas; and a method in which an oxygen adsorber is accommodated in the packaging article.

Examples of the inert gas can include: rare gases such as helium gas, neon gas, and argon gas; nitrogen gas; and the like.

Examples of the oxygen adsorber can include AGELESS (commercially available product) which is an iron-based oxygen adsorber, and the like.

The packaging article for accommodating the medical rubber product may not be particularly limited, for instance, as long as the packaging article can be irradiated with the gamma ray. Examples of the form of the packaging article can include the forms of a bag, a box, and the like. Examples of the packaging bag can include packaging bags formed of aluminum or a thermoplastic resin film made from polyethylene, polyamide, or polyester. The packaging bag can be preferably one that can be sealed. The packaging box may not be particularly limited, and examples of the packaging box can include a box made from paper, a box made from cardboard, and the like.

Examples of the packaging article can include: a packaging article having gas permeability; and a packaging article having non-gas permeability (gas sealability). It may also be preferable to use these packaging articles in combination.

Irradiation of the medical rubber product with the gamma ray may be performed on, for example, a packaging article (for example, a cardboard box) further accommodating a plurality of primary packaging articles (for example, packaging bags) accommodating a plurality of the medical rubber products.

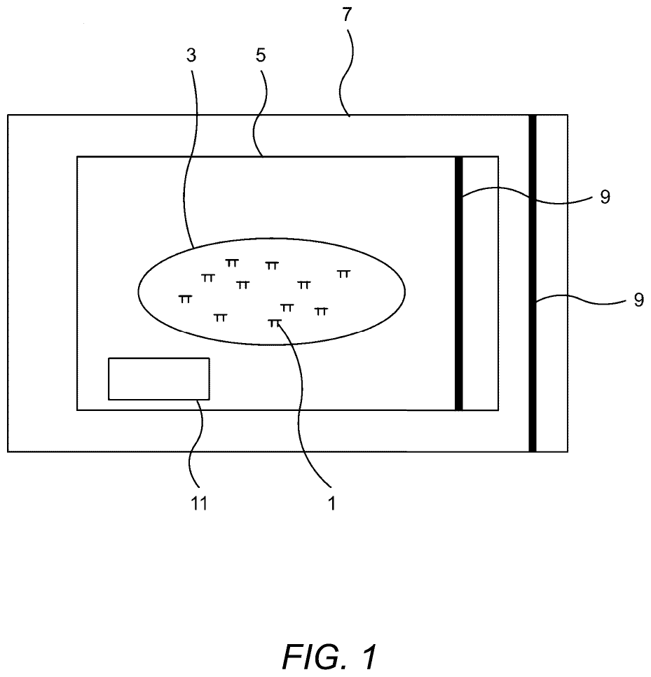

FIG. 1 is a diagram for schematically explaining an example of a packaging mode for irradiation with the gamma ray. In the mode shown in FIG. 1, a primary packaging article 3 accommodating a plurality of medical rubber products 1 can be further accommodated in a secondary static charge prevention packaging article 5 and a tertiary static charge prevention packaging article 7. As the primary packaging article 3, a packaging article having gas permeability can be preferable. As the secondary static charge prevention packaging article 5 and the tertiary static charge prevention packaging article 7, packaging articles capable of sealing gas therein can be preferable. Each of the secondary static charge prevention packaging article 5 and the tertiary static charge prevention packaging article 7 can be preferably sealed with a heat seal 9. In the case of using an oxygen adsorber 11, the oxygen adsorber 11 can be preferably disposed between the primary packaging article 3 and the secondary packaging article 5 such that the oxygen adsorber 11 does not come into direct contact with the medical rubber products 1. By disposing the oxygen adsorber 11 in the secondary packaging article 5, the oxygen concentration in each of the secondary packaging article 5 and the primary packaging article 3 can be set to be not higher than 5%. Irradiation with the gamma ray can be performed with a plurality of the tertiary static charge prevention packaging articles 7 being accommodated in a quaternary packaging article (for example, a cardboard box).

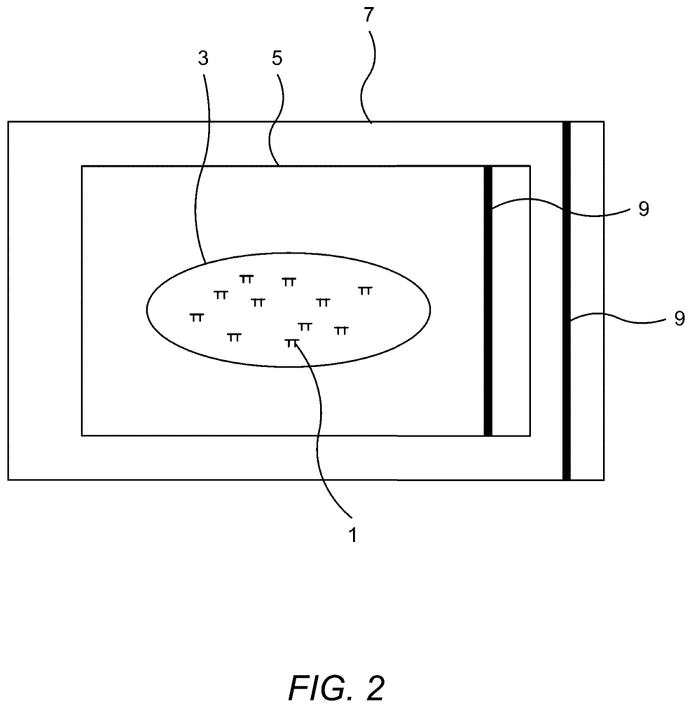

FIG. 2 is a diagram for schematically explaining another example of the packaging mode for irradiation with the gamma ray. In the mode shown in FIG. 2, the primary packaging article 3 accommodating the plurality of medical rubber products 1 can be further accommodated in the secondary static charge prevention packaging article 5 and the tertiary static charge prevention packaging article 7. Each of the secondary static charge prevention packaging article 5 and the tertiary static charge prevention packaging article 7 can be preferably sealed with the heat seal 9. As the primary packaging article 3, a packaging article having gas permeability can be preferable. As the secondary static charge prevention packaging article 5 and the tertiary static charge prevention packaging article 7, packaging articles capable of sealing gas therein can be preferable. The secondary packaging article 5 accommodating the primary packaging article 3 can be filled with an inert gas. The filling with the inert gas can make it possible to set the oxygen concentration in each of the secondary packaging article 5 and the primary packaging article 3 to be not higher than 5%, as an example. Irradiation with the gamma ray can be performed with a plurality of the tertiary static charge prevention packaging articles 7 being accommodated in a quaternary packaging article (for example, a cardboard box).

At the time of irradiation with the gamma ray, irradiation with the gamma ray can be preferably performed in a state where the packaging article accommodating the plurality of medical rubber products is accommodated in, for example, an accommodation container made from an aluminum alloy.

The viable cell count (cfu: colony forming unit (the number of colonies that appear under incubation)), in a bioburden measurement test, of the sterilized medical rubber product according to one or more embodiments of the present invention can be preferably 0.

Examples of the medical rubber product according to one or more embodiments of the present disclosure can include: rubber plugs and sealing members of containers (for example, vials) for various medical preparations such as a liquid preparation, a powder preparation, and a freeze-dried preparation; slidable parts and sealing parts such as rubber plugs for vacuum blood collection tubes, and plunger stoppers and nozzle caps for pre-filled syringes; and the like.

### EXAMPLES

Hereinafter, one or more embodiments of the present disclosure will be described in detail by means of examples, but embodiments of the present disclosure are not limited to the following examples, and any of modifications and implementation modes made within the scope of the gist of the present disclosure is included in the scope of embodiments of the present disclosure.

### [Preparation of Medical Rubber Composition]

A rubber composition was prepared by blending components other than the crosslinking component (triazine-based crosslinking agent) among the components indicated in Table 1, kneading the resultant mixture with use of a 10-L pressurization-type sealed kneader at a filling rate of 75%, aging the kneaded product at room temperature, then, adding the crosslinking component to the kneaded product, and kneading the resultant mixture with use of an open roll.

TABLE 1

| | Blending amount |
|---|---|
| Medical rubber composition | (parts by mass) |
| Chlorinated isobutylene-isoprene rubber | 100 |
| Polyethylene | 5 |
| Triazine-based crosslinking agent | 1 |
| Talc | 50 |
| Hydrotalcite | 2 |
| Magnesium oxide | 2 |
| Carbon | 0.5 |
| Titanium oxide | 3 |
| Oil | 3 |

Details of the blending materials used are as follows.

Chlorinated isobutylene-isoprene rubber: 1066 (chlorine content: 1.26% by weight) manufactured by Exxon Mobil Corporation

Polyethylene: MIPELON XM-220 (degree of crystallinity: 69%) manufactured by Mitsui Chemicals, Inc.

Triazine derivative: ZISNET DB manufactured by SANKYO KASEI CO., LTD.

Talc: MISTRON Vapor manufactured by Imerys Specialties Japan Co., Ltd.

Hydrotalcite: ALCAMIZER 1 manufactured by Kyowa Chemical Industry Co., Ltd.

Magnesium oxide: MAGSARAT 150s manufactured by Kyowa Chemical Industry Co., Ltd.

Carbon black: DIABLACK G manufactured by Mitsubishi Chemical Holdings Corporation

Titanium oxide: KR-380 manufactured by Titan Kogyo Ltd.

Oil: PW380 manufactured by Idemitsu Kosan Co., Ltd.

### [Manufacturing of Medical Rubber Plugs]

The aforementioned rubber composition was molded in the shape of a sheet, sandwiched between an upper mold and a lower mold, and press-molded in a vacuum at 180° C. for 6 minutes, and a plurality of rubber plugs for vials for a freeze-dried injectable were continuously formed on the above one sheet. In each rubber plug, a flange had a diameter of 19.0 mm, the total height was 13.0 mm, a leg portion had a diameter of 7.20 mm, and a flange piercing portion had a thickness of 2.5 mm. Thereafter, a silicone-based lubricating coat agent was applied on both surfaces of the above sheet. Then, rubber plugs were manufactured through an outer appearance inspection step, a stamping step, a cleaning step, a sterilizing step, and a drying step. Each of the manufactured rubber plugs was used in an eluting-substance test and a tack test. Conditions of sterilization with the gamma ray are indicated in Table 2.

TABLE 2

| Sterilization No. | | 1 | 2 | 3 | 4 | 5 | 6 | 7 |
|---|---|---|---|---|---|---|---|---|
| Conditions of | Packaging-article | Oxygen | Oxygen | Oxygen | Oxygen | Air | Air | Air |
| irradiation | internal | adsorber | adsorber | adsorber | adsorber | | | |
| with gamma | environment | contained | contained | contained | contained | | | |
| ray | Oxygen | 0.1% | 0.1% | 0.1% | 0.1% | 21% | 21% | 21% |
| | concentration in | | | | | | | |
| | packaging article (%) | | | | | | | |
| | Absorbed dose (kGy) | 15 | 25 | 50 | 100 | 50 | 100 | 200 |
| Analysis with | (Ss/Si) × 100 | 101 | 105 | 102 | 110 | 201 | 270 | 393 |
| FT-IR | Ss = (As/Bs) × 100 | 3.9 | 4.0 | 3.9 | 4.2 | 7.7 | 10.3 | 15.0 |
| | Si = (Ai/Bi) × 100 | 3.8 | 3.8 | 3.8 | 3.8 | 3.8 | 3.8 | 3.8 |
| (1) Eluting- | Overall evaluation | Pass | Pass | Pass | Pass | Pass | Pass | Not |
| substance test | | | | | | | | pass |
| (2) TOC eluting- | Relative evaluation | 93% | 100% | 119% | 129% | 398% | 783% | 1831% |
| substance test | (after irradiation/ | A | A | A | B | C | C | C |
| | before irradiation) | | | | | | | |
| (3) Tack test | Relative evaluation | 95% | 96% | 102% | 118% | 355% | 803% | 1430% |
| | (after irradiation/ | A | A | A | A | C | C | C |
| | before irradiation) | | | | | | | |
| (4) Viable cell | Test result (n = 5) | 0 | 0 | 0 | 0 | 0 | 0 | 0 |
| count (cfu) | | | | | | | | |

| | | | | | | | |
|---|---|---|---|---|---|---|---|
| Overall determination as to suitability | Suitable | Suitable | Suitable | Suitable | Un- | Un- | Un- |
| for being ready-to-use | | | | | suitable | suitable | suitable |

### [Evaluation Methods]

(1) Measurement with Fourier Transform Infrared Spectrophotometer (FT-TR)

A measurement sample was made by being cut out from the upper surface of each of the sterilized medical rubber plugs with use of a razor so as to have a thickness (about 1.0 mm) that allowed the measurement sample to be placed on a measurement device. The medical rubber plug was cut in the longitudinal direction through the center thereof in a plan view, and a measurement sample was obtained by being cut out from a center portion of the obtained cross section with use of the razor so as to have a thickness (for example, 0.5 mm to 2.0 mm) that allowed the measurement sample to be placed on the measurement device. Each of the measurement samples was placed on the measurement device, and a region, of a surface portion of the measurement sample, having an outer diameter of 1 mm was irradiated with light and was subjected to measurement.

FT-TR measurement was performed a total of 16 times at a wave number resolution of 4 cm⁻¹ by using Frontier with a MIRacle ATR unit (Ge) manufactured by PerkinElmer Co., Ltd.

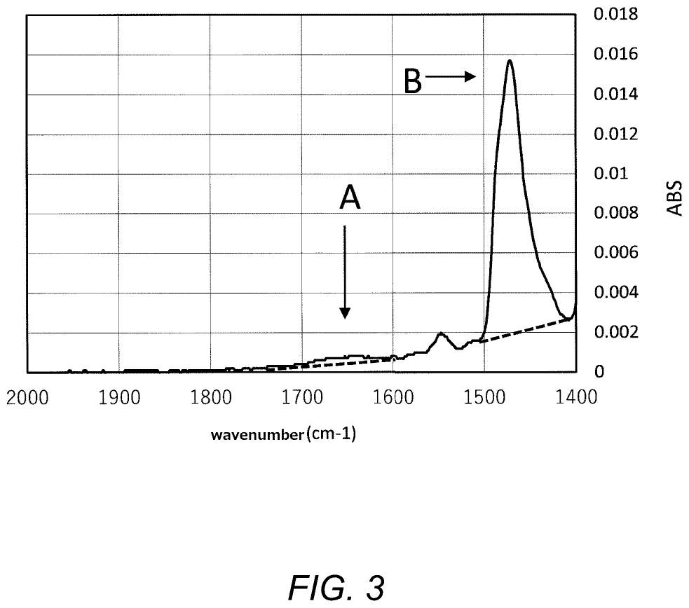

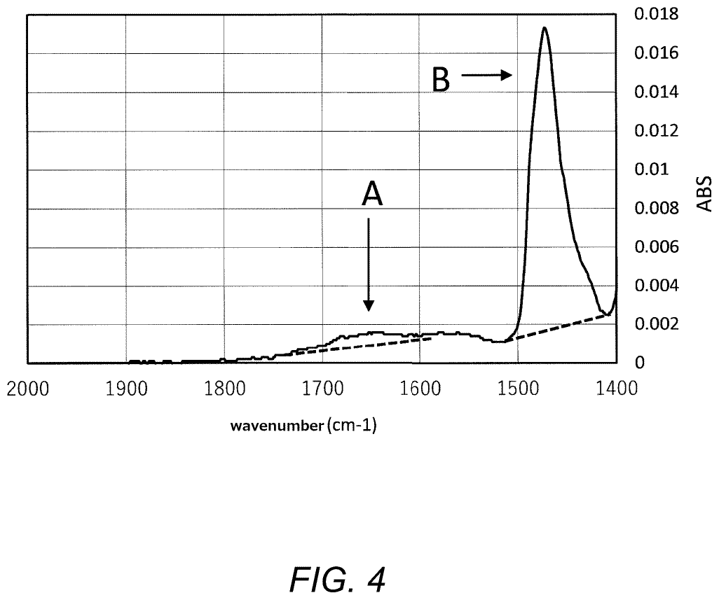

FIG. 3 is an FT-JR chart of the surface portion of a medical rubber product having been subjected to sterilization No. 1. FIG. 4 is an FT-IR chart of the surface portion of a medical rubber product having been subjected to sterilization No. 7.

In each of the obtained infrared absorption spectra, an infrared absorption peak A having an absorption peak of absorbance at around a wave number of 1650 cm⁻¹, and an infrared absorption peak B having an absorption peak of absorbance at around a wave number of 1470 cm⁻¹, are present. For the infrared absorption peak A, a baseline was drawn between 1750 cm⁻¹ and 1590 cm⁻¹, and an area (As) delimited by the baseline was calculated. For the infrared absorption peak B, a baseline was drawn between 1510 cm⁻¹ and 1408 cm⁻¹, and an area (Bs) delimited by the baseline was calculated. Regarding the inside of the medical rubber plug as well, an area (Ai) of the infrared absorption peak A and an area (Bi) of the infrared absorption peak B were calculated in the same manner.

### (2) Measurement of Melting Heat Quantity of Polyethylene

The melting heat quantity of the polyethylene was acquired from a primary test at elevated temperatures through differential scanning calorimetry (DSC).

DSC measurement condition: 20° C. to 200° C., with the speed of temperature elevation being 10° C./min.

### (3) Eluting-Substance Test

Measurement sample: in a state where each of the medical rubber plugs was put into a polyethylene bag (in either of the packaging modes shown in FIG. 1 and FIG. 2), the medical rubber plug was irradiated with the gamma ray in the corresponding packaging-article internal environment indicated in Table 2 so as to have the corresponding absorbed dose indicated in Table 2. Consequently, a rubber plug having been irradiated with the gamma ray was obtained.

The measurement sample was tested according to the method in “Extractable substances” described in “7.03 Test for Rubber Closure for Aqueous Infusions” of the 17th edition of the Japanese Pharmacopoeia. Conditions of passing the test were as follows.

### Properties of Test Solution: Colorless and Clear

Ultraviolet transmissivity: a transmissivity being not lower than 99.0% at each of a wavelength of 430 nm and a wavelength of 650 nm with a layer length of 10 mm

Ultraviolet absorption spectrum: an absorbance being not higher than 0.20 at a wavelength of 220 nm to 350 nm

pH: the difference between the test solution and a blank test solution being not larger than 1.0

Zinc: the absorbance of a sample solution being not higher than the absorbance of a standard solution

Potassium permanganate reducing substance: not higher than 2.0 mL/100 mL (according to a standard in the Japanese Pharmacopoeia)

Post-evaporation residue: not larger than 2.0 mg

If any of these conditions was not satisfied, the measurement sample was evaluated as “Not pass”. Meanwhile, if all of the conditions were satisfied, the measurement sample was evaluated as “Pass”.

### (4) TOC Test

Regarding the eluting liquid subjected to the eluting-substance test in “(3)”, a total organic carbon value TOC was measured (through a non-purgeable organic carbon (NPOC) method).

Measurement analysis device: a Shimadzu total organic carbon analyzer TOC-LCPH (of a combustion oxidizing type)

Measurement analysis condition: a combustion tube temperature being 680 degrees with use of a high-sensitivity catalyst

Carrier gas: highly purified air at 150 mL/min.

Injection amount: 150 μL

Concentration of added acid: 1%

Aeration treatment time: 60 sec.

Eluting characteristics obtained before and after irradiation with the gamma ray were evaluated. The TOC obtained after irradiation with the gamma ray as a relative index was evaluated with the TOC obtained before irradiation with the gamma ray being regarded as 100%. A larger numeral means that the eluting performance has further deteriorated relative to the eluting performance obtained before irradiation with the gamma ray.

### Evaluation Criteria

A: not higher than 125% (equivalent to a pre-irradiation numeral)
B: higher than 125% and not higher than 150% (having slightly deteriorated relative to the pre-irradiation numeral)
C: higher than 150% (having deteriorated to a large extent, relative to the pre-irradiation numeral)

### (5) Bioburden Measurement Test

As a bioburden measurement method, a method in which ultrasonic recovery and culture medium immersion are performed in combination was employed.

### Operation Procedure

a: Samples were transferred to test tubes (φ18 mm×180 mm) in which 10 mL of PTS recovery liquids (1% of peptone, 0.1% of Tween 80, and 0.85% of saline) had been respectively dispensed.
b: Ultrasonication was performed for 15 minutes.
c: Each of the recovery liquids was suctioned and filtered through a Milliflex membrane filter (MF).
d: The post-filtering Milliflex membrane filter (MF) was pasted on an SCDA (soybean-casein digest agar) plate culture medium.
e: The post-ultrasonication sample was transferred onto a deep petri dish and was immersed in an SCDA culture medium containing 0.002% of triphenyltetrazolium chloride.
f: The sample was cultured at 30° C. to 35° C. for seven days.
g: The culture medium after the culturing for seven days was observed, and the total of a viable cell count obtained through ultrasonic recovery and a viable cell count obtained through culture medium immersion was used as a viable cell count (cfu). The viable cell count (cfu) is the average value of viable cell counts with respect to five samples of each type.

### (6) Tack Test

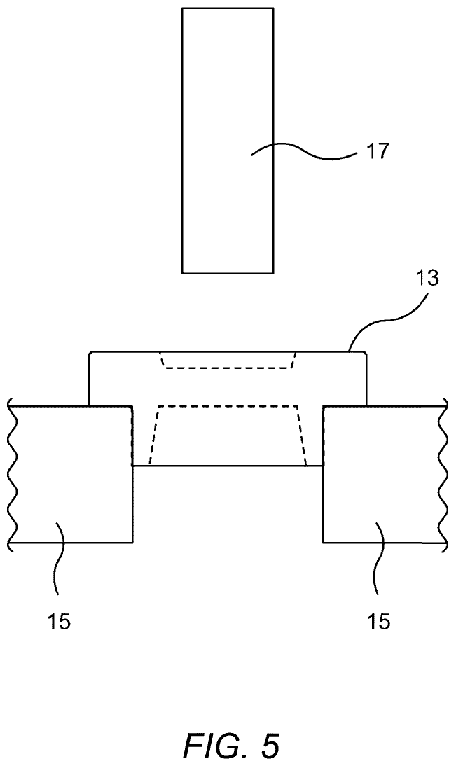

A tack test was performed as follows by using a desktop tester EZ-SX available from Shimadzu Corporation. That is, as shown in FIG. 5, a sample 13 was fixed to fixation jigs 15 on the lower side, a metal probe 17 on the upper side was pressed onto the sample 13, and the pressed state was maintained for 10 seconds after the pressure had reached a set value. Thereafter, the metal probe 17 was lifted upward, and a peak value of close contact force generated between the metal probe 17 and the sample 13 was used as a tack value. The measurement was performed 5 times on each sample. From among the measurement values obtained as a result, the maximum value and the minimum value were excluded, and the average value of the remaining three measurement values was calculated. The close contact force of the sample having been irradiated with the gamma ray was indicated as an index with the close contact force of the sample, which had not yet been irradiated with the gamma ray, being regarded as 100. A smaller index indicates a lower adhesiveness and a more favorable result.

### Measurement Conditions

Pressing speed: 0.5 mm/s

Pressing load: 1000 g of weight

Pressed-state maintaining time: 10 seconds

Pull-up speed: 10 mm/s

Ultimate pull-up distance: 3 mm

Diameter of probe: 10 mm

### Evaluation Criteria

A: not higher than 120% (equivalent to a pre-irradiation value)
B: higher than 120% and not higher than 150% (having slightly deteriorated relative to the pre-irradiation value)
C: higher than 150% (having deteriorated to a large extent, relative to the pre-irradiation value)

The results of the eluting-substance test, the TOC test, and the tack test are indicated together in Table 2.

Determination as to suitability for being ready-to-use was performed as follows.

If the result of the eluting-substance test was “Pass”, the result of the TOC test was B or the more favorable evaluation result, the result of the tack test was B or the more favorable evaluation result, and the viable cell count (cfu) was zero, it was determined that suitability for being ready-to-use was attained. Meanwhile, if any one of the evaluation results was unsatisfactory, it was determined that suitability for being ready-to-use was not attained.

According to the results indicated in Table 2, non-eluting characteristics are maintained and less troubles occur in a medical product manufacturing process, in the case of a medical rubber product formed from an elastic material and sterilized by being irradiated with gamma ray or electron ray (beta ray), wherein, when measurement is performed on a surface portion of the elastic material through attenuated total reflection (ATR) by using a Fourier transform infrared spectrophotometer (FT-IR), if an area of an infrared absorption peak at around a wave number of 1650 cm⁻¹ in an infrared absorption spectrum obtained through the measurement is defined as As and an area of an infrared absorption peak at around a wave number of 1470 cm⁻¹ in the infrared absorption spectrum is defined as Bs, Ss=(As/Bs)×100≤6 is satisfied.

The present invention makes it possible to provide a medical rubber product in which non-eluting characteristics are maintained even after sterilization with gamma ray and with which less troubles occur in a medical product manufacturing process.

Preferable mode (1) of the present disclosure can be directed to a medical rubber product formed from an elastic material and sterilized by being irradiated with gamma ray or electron ray (beta ray), wherein, when measurement is performed on a surface portion of the elastic material through attenuated total reflection (ATR) by using a Fourier transform infrared spectrophotometer (FT-IR), if an area of an infrared absorption peak at around a wave number of 1650 cm⁻¹ in an infrared absorption spectrum obtained through the measurement is defined as As and an area of an infrared absorption peak at around a wave number of 1470 cm⁻¹ in the infrared absorption spectrum is defined as Bs, Ss=(As/Bs)×100≤6 is satisfied.

Preferable mode (2) of the present disclosure can be directed to the medical rubber product according to mode (1), wherein, when cut-out from a center portion of the medical rubber product is performed and measurement is performed on an infrared absorption spectrum of an exposed inside of the medical rubber product, if an area of an infrared absorption peak at around a wave number of 1650 cm⁻¹ in the infrared absorption spectrum is defined as Ai, an area of an infrared absorption peak at around a wave number of 1470 cm⁻¹ in the infrared absorption spectrum is defined as Bi, and Si=(Ai/Bi)×100 is satisfied, P=(Ss/Si)×100≤150 is satisfied on the inside.

Preferable mode (3) of the present disclosure can be directed to the medical rubber product according to mode (1) or (2), wherein the elastic material is a cured product of a rubber composition containing: a (a) base polymer containing a halogenated isobutylene-isoprene rubber; and a triazine derivative as a (c) crosslinking agent.

Preferable mode (4) of the present disclosure can be directed to the medical rubber product according to mode (3), wherein the halogenated isobutylene-isoprene rubber is at least one type of rubber selected from the group consisting of chlorinated isobutylene-isoprene rubbers, brominated isobutylene-isoprene rubbers, and brominated isobutylene-para-methylstyrene copolymer rubbers.

Preferable mode (5) of the present disclosure can be directed to the medical rubber product according to mode (3) or (4), wherein the rubber composition further contains a (b) polyethylene.

Preferable mode (6) of the present disclosure can be directed to the medical rubber product according to mode (5), wherein the (b) polyethylene contains a polyethylene having a degree of crystallinity not higher than 70%.

Preferable mode (7) of the present disclosure can be directed to the medical rubber product according to mode (5) or (6), wherein an amount of the (b) polyethylene contained per 100 parts by mass of the (a) base polymer is not smaller than 3 parts by mass and not larger than 30 parts by mass.

Preferable mode (8) of the present disclosure can be directed to the medical rubber product according to any one of modes (1) to (7), wherein the elastic material has a JIS-A hardness of 30 to 70 degrees and a compression set not higher than 20%, the compression set being measured under conditions of 70° C., 22 hours, and 25% according to JIS K 6262.

Preferable mode (9) of the present disclosure can be directed to the medical rubber product according to any one of modes (1) to (8), the medical rubber product being a rubber plug for a vial, a cap or a plunger stopper for a syringe, or a rubber plug for a vacuum blood collection tube.

What is claimed is:

1. A medical rubber product formed from an elastic material and sterilized by being irradiated with gamma ray or electron ray (beta ray), wherein, upon measurement being performed on a surface portion of the elastic material through attenuated total reflection (ATR) by using a Fourier transform infrared spectrophotometer (FT-IR), under a condition that an area of an infrared absorption peak at around a wave number of 1650 cm⁻¹ in an infrared absorption spectrum obtained through the measurement is defined as As and an area of an infrared absorption peak at around a wave number of 1470 cm⁻¹ in the infrared absorption spectrum is defined as Bs, Ss=(As/Bs)×100≤6 is satisfied.

2. The medical rubber product according to claim 1, wherein, upon cut-out from a center portion of the medical rubber product is performed and measurement is performed on an infrared absorption spectrum of an exposed inside of the medical rubber product, under the condition that the area of the infrared absorption peak at around the wave number of 1650 cm⁻¹ in the infrared absorption spectrum is defined as Ai, the area of an infrared absorption peak at around the wave number of 1470 cm⁻¹ in the infrared absorption spectrum is defined as Bi, and Si=(Ai/Bi)×100 is satisfied, P=(Ss/Si)×100≤150 is satisfied on the inside.

3. The medical rubber product according to claim 1, wherein the elastic material is a cured product of a rubber composition containing: a (a) base polymer containing a halogenated isobutylene-isoprene rubber; and a triazine derivative as a (c) crosslinking agent.

4. The medical rubber product according to claim 3, wherein the halogenated isobutylene-isoprene rubber is at least one type of rubber selected from the group consisting of chlorinated isobutylene-isoprene rubbers, brominated isobutylene-isoprene rubbers, and brominated isobutylene-para-methylstyrene copolymer rubbers.

5. The medical rubber product according to claim 3, wherein the rubber composition further contains a (b) polyethylene.

6. The medical rubber product according to claim 5, wherein the (b) polyethylene contains a polyethylene having a degree of crystallinity not higher than 70%.

7. The medical rubber product according to claim 5, wherein an amount of the (b) polyethylene contained per 100 parts by mass of the (a) base polymer is not smaller than 3 parts by mass and not larger than 30 parts by mass.

8. The medical rubber product according to claim 1, wherein the elastic material has a JIS-A hardness of 30 to 70 degrees and a compression set not higher than 20%, the compression set being measured under conditions of 70° C., 22 hours, and 25% according to JIS K 6262.

9. The medical rubber product according to claim 1, the medical rubber product being a rubber plug for a vial, a cap or a plunger stopper for a syringe, or a rubber plug for a vacuum blood collection tube.
